(19) United States
(12) Patent Application Publication
(10) Pub. No.: US 2019/0246879 A1
(43) Pub. Date: Aug. 15, 2019

(54) ENDOSCOPE SYSTEM

(71) Applicant: OLYMPUS CORPORATION, Tokyo (JP)

(72) Inventor: Masahiro KATAKURA, Tokyo (JP)

(73) Assignee: OLYMPUS CORPORATION, Tokyo (JP)

(21) Appl. No.: 16/394,616

(22) Filed: Apr. 25, 2019

Related U.S. Application Data
(63) Continuation of application No. PCT/JP2017/046079, filed on Dec. 22, 2017.

(30) Foreign Application Priority Data
Dec. 26, 2016 (JP) 2016-251392

(51) Int. Cl. A61B 1/00; A61B 1/045; G02B 13/04; G02B 23/26

(52) U.S. Cl. CPC A61B 1/00096; A61B 1/00009; A61B 1/00163; A61B 1/0676; G02B 13/04; G02B 23/26; A61B 1/045

(57) ABSTRACT

An endoscope system includes an objective optical system, an optical-path splitter which acquires two optical images with different focus, an image sensor which acquires the optical images. The objective optical system includes a first lens group having a negative refractive power, a second lens group having a positive refractive power, and a third lens group having a positive refractive power. Switching to a normal observation and a close observation is possible by moving the second lens group toward an image side. The first lens group includes a first lens having a negative refractive power, and at least one positive lens, and the following conditional expressions (1)′″, (2), and (3)′″ are satisfied:

1.40≥D_2T/fw<5 (1)′″

1.01<ω(wide)/ω(tele)<3.0 (2)

2.70≤D_1G/D_2T<3.21 (3)′″.

### CROSS-REFERENCE TO RELATED APPLICATION

The present application is a continuation application of PCT/JP2017/046079 filed on Dec. 22, 2017 which is based upon and claims the benefit of priority from Japanese Patent Application No. 2016-251392 filed on Dec. 26, 2016; the entire contents of which are incorporated herein by reference.

### BACKGROUND OF THE INVENTION

### Field of the Invention

The present invention relates to an endoscope system.

### Description of the Related Art

Generally, in an equipment such as an endoscope system which includes an image pickup element, it has been known that a depth of field becomes narrow as the number of pixels of the image pickup element is made large. In other words, in the image pickup element, when a pixel pitch (a vertical and horizontal dimension of one pixel) is made small in order to increase the number of pixels, since a permissible circle of confusion also becomes small in association with narrowing the pixel pitch, the depth of field of an image pickup apparatus becomes narrow.

For widening the depth of field, an arrangement in which a self-image is split and images are formed by using an optical-path splitting prism, and the images acquired are combined by image processing and the depth is widened, has been known. For disposing the optical-path splitting prism, a long rear focal length (back focus, hereinafter, appropriately referred to as ‘fb’) is necessary.

As an optical system having a long fb, optical systems disclosed in Japanese Patent Publication No. 6006464 and Japanese Patent Application Laid-open Publication No. 2007-093961 have been known.

The optical system disclosed in Japanese Patent Publication No. 6006464 includes in order from an object side, a first lens group having a negative refractive power, a second lens group having a positive refractive power, and a third lens group having a positive refractive power. Accordingly, the optical system is superior at a point of capability of shortening a total length of the optical system and acquiring a long fb while having a fewer number of lenses. Furthermore, the optical system is also superior at a point of enabling a close observation and a normal observation by moving the second lens group having a positive refractive power in an optical axial direction.

The optical system disclosed in Japanese Patent Application Laid-open Publication No. 2007-093961 includes in order from an object side, a first lens group having a negative refractive power, a second lens group having a positive refractive power, and a third lens group having a positive refractive power. Accordingly, the optical system is superior at a point of capability of shortening a total length of the optical system and acquiring a long fb while having a fewer number of lenses. Furthermore, since the first lens group is fixed at the time of zooming, the optical system is favorable at a point of being strong against reduction in the number of components, and a manufacturing variation.

### SUMMARY OF THE INVENTION

An endoscope system according to at least some embodiments of the present invention comprises,

an objective optical system,

an optical-path splitter which splits an object image acquired by the objective optical system into two optical images with different focus by using two prisms, and

an image sensor which acquires the optical images,

wherein

the objective optical system includes in order from the object side to an image side,

a first lens group having a negative refractive power, which is fixed,

a second lens group having a positive refractive power, which is movable,

a third lens group having a positive refractive power, which is fixed, and

switching to a normal observation and a close observation (magnified observation) is possible by moving the second lens group toward an image side,

the first lens group includes in order from the object side to the image side, a first lens having a negative refractive power, and at least one positive lens, and

the objective optical system satisfies the following conditional expressions (1)′″, (2), and (3)′″:

1.40≤D_2T/fw<5 (1)′″

1.01<ω(wide)/ω(tele)<3.0 (2)

2.70≤D_1G/D_2T<3.21 (3)′″

where

D_2T denotes a thickness on an optical axis of the positive lens nearest to an image in the first lens group having a negative refractive power,

fw denotes a focal length of the objective optical system in a normal observation state,

ω(wide) denotes an angle of view of the objective optical system in the normal observation state,

ω(tele) denotes an angle of view of the objective optical system in a close observation state,

D_1G denotes a thickness on the optical axis of the first lens group having a negative refractive power, and

D_2T denotes a thickness on the optical axis of the positive lens nearest to the image in the first lens group having a negative refractive power.

### BRIEF DESCRIPTION OF THE DRAWINGS

FIG. 1 is a cross-sectional view of an arrangement of an objective optical system, an optical-path splitter, and an image pickup element in an endoscope system according to an embodiment of the present invention;

FIG. 2A and FIG. 2B are cross-sectional views of an arrangement of an objective optical system, an optical-path splitter, and an image pickup element in an endoscope system according to an example 1 of the present invention, where, FIG. 2A is a cross-sectional view in a normal observation state, and FIG. 2B is a cross-sectional view in a close observation state;

FIG. 3A, FIG. 3B, FIG. 3C, FIG. 3D, FIG. 3E, FIG. 3F, FIG. 3G, and FIG. 3H are aberration diagrams of the example 1;

FIG. 4A and FIG. 4B are cross-sectional views of an arrangement of an objective optical system, an optical-path splitter, and an image pickup element in an endoscope system according to an example 2 of the present invention, where, FIG. 4A is a cross-sectional view in a normal observation state, and FIG. 4B is a cross-sectional view in a close observation state;

FIG. 5A, FIG. 5B, FIG. 5C, FIG. 5D, FIG. 5E, FIG. 5F, FIG. 5G, and FIG. 5H are aberration diagrams of the example 2;

FIG. 6A and FIG. 6B are cross-sectional views of an arrangement of an objective optical system, an optical-path splitter, and an image pickup element in an endoscope system according to an example 3 of the present invention, where, FIG. 6A is a cross-sectional view in a normal observation state, and FIG. 6B is a cross-sectional view in a close observation state;

FIG. 7A, FIG. 7B, FIG. 7C, FIG. 7D, FIG. 7E, FIG. 7F, FIG. 7G, and FIG. 7H are aberration diagrams of the example 3;

FIG. 8 is a schematic block diagram of an optical-path splitter and an image pickup element in an endoscope system according to an embodiment of the present invention;

FIG. 9 is a schematic block diagram of the image pickup element in the endoscope system according to the embodiment of the present invention;

FIG. 10 is a functional block diagram showing an arrangement of the endoscope system according to the embodiment of the present invention;

FIG. 11 is a flowchart showing a flow in a case in which two optical images are combined in the endoscope system according to the embodiment of the present invention; and

FIG. 12 is diagram showing an image-formation state in a case in which an image is formed on an image pickup element after reflection for odd number of times by a beam splitter in the endoscope system according to the embodiment of the present invention.

### DETAILED DESCRIPTION OF THE INVENTION

Reasons for and effects of adopting the following arrangement for an endoscope system 10 (FIG. 10) according to the present embodiment will be described below by using the accompanying diagrams. However, the present invention is not restricted to the embodiment described below.

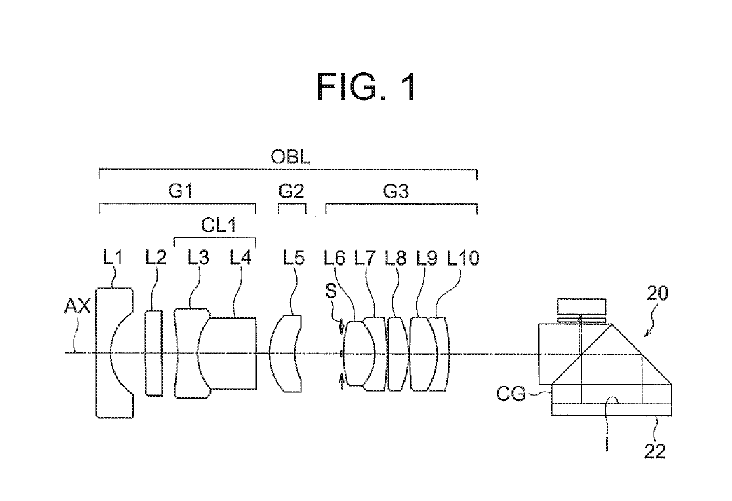

An endoscope system according to the present embodiment, as shown in FIG. 1, includes an objective optical system OBL, an optical-path splitter 20 which splits an object image acquired by the objective optical system OBL into two optical images with different focus by using two prisms, an image pickup element 22 which acquires the optical image, and an image combining processing section 23c (FIG. 10) which selects in a predetermined area, an image with a relatively high contrast out of two optical images acquired, and generates a combined image.

The objective optical system OBL includes in order from an object side, a first lens group G1 having a negative refractive power, which is fixed, a second lens group G2 having a positive refractive power, which is movable, and a third lens group G3 having a positive refractive power, which is fixed.

In the objective optical system OBL, switching to a normal observation (distant observation) and a close observation (magnified observation) is possible by moving the second lens group G2 toward an image side.

The first lens group G1 includes in order from the object side, a first lens L1 having a negative refractive power, and at least one positive lens L4.

The endoscope system according to the present embodiment has the objective optical system which satisfies the following conditional expressions (1) and (2):

1.3<D_2T/fw<5 (1)

1.01<ω(wide)/ω(tele)<3.0 (2)

where,

D_2T denotes a thickness on an optical axis AX of the positive lens L4 nearest to an image in the first lens group G1 having a negative refractive power,

fw denotes a focal length of the objective optical system OBL in a normal observation state,

ω(wide) denotes an angle of view of the objective optical system OBL in the normal observation state, and

ω(tele) denotes an angle of view of the objective optical system OBL in a close observation state.

In the present embodiment, the objective optical system includes in order from the object side, the first lens group G1 having a negative refractive power, the second lens group G2 having a positive refractive power which is movable at a time of focusing, and the third lens group G3 having a positive refractive power. Accordingly, it is possible to switch to the normal observation and the close observation, and it is possible to secure a long back focus. Moreover, it is possible to achieve an optical system in which fluctuation of aberration at the time of focusing is small, and an optical system which is strong against a manufacturing error. Furthermore, as it will be described later, by acquiring two optical images having different focus and generating a combined image, it becomes possible to achieve an image having a wide depth of field.

Conditional expression (1) is related to an appropriate ratio of D_2T and fw. The conditional expression (1) is a conditional expression related to at least one positive lens in the first lens group G1 having a negative refractive power. Within a range satisfying conditional expression (1), it is possible to correct a curvature of field favorably while securing the long back focus. For this, it is preferable to satisfy conditional expression (1).

In a case of exceeding an upper limit value of conditional expression (1) or falling below a lower limit value of conditional expression (1), the curvature of field occurs largely. Therefore, it is not preferable to exceed the upper limit value of conditional expression (1), and to fall below the lower limit value of conditional expression (1).

Conditional expression (2) is related to an appropriate ratio of ω(wide) and ω(tele). Moreover, conditional expression (2) is a conditional expression which indicates a variation in an angle of view at the time of focusing. Within a range satisfying conditional expression (2), it is possible to achieve an appropriate variation in the angle of view.

In a case of falling below a lower limit value of conditional expression (2), an acceptable amount of the variation in the angle of view at the time of focusing becomes small. In an optical system which includes a first lens group G1 having a negative refractive power, a second lens group G2 having a positive refractive power, and a third lens group G3 having a positive refractive power, it is not possible to realize a desired optical system. Therefore, it is not preferable to fall below the lower limit value of conditional expression (2).

In a case of exceeding an upper limit value of conditional expression (2), the variation in the angle of view is excessively large. Therefore, it is necessary to impart a large power (refractive power) to the second lens group G2 having a positive refractive power. As a result, the optical system becomes weak against the manufacturing error. Therefore, it is not preferable to exceed the upper limit value of conditional expression (2).

It is preferable that the following conditional expression (1)′ be satisfied instead of conditional expression (1).

1.6<D_2T/fw<2.5 (1)′

Furthermore, it is more preferable that the following conditional expression (1) ” be satisfied instead of conditional expression (1).

1.9<D_2T/fw<2.0 (1)′

It is preferable that the following conditional expression (2)′ be satisfied instead of conditional expression (2).

1.01<ω(wide)/ω(tele)<2.0 (2)

Furthermore, it is more preferable that the following conditional expression (2)′ be satisfied instead of conditional expression (2).

1.01<ω(wide)/ω(tele)<1.1 (2)′

Moreover, according to a preferable aspect of the present embodiment, it is preferable that the following conditional expression (3) be satisfied:

1.5≤D_1G/D_2T<3.21 (3)

where,

D_1G denotes a thickness on an optical axis AX of the first lens group G1 having a negative refractive power, and

D_2T denotes the thickness on the optical axis AX of the positive lens L4 nearest to the image in the first lens group G1 having a negative refractive power.

Conditional expression (3) is related to an appropriate ratio of D_1G and D_2T. Moreover, conditional expression (3) is a conditional expression related to the thickness on the optical axis AX of the first lens group G1 having a negative refractive power and the thickness of the positive lens L4 nearest to the image in the first lens group G1.

Within a range satisfying conditional expression (3), D_1G and D_2T have values such that the ratio thereof becomes an appropriate value. In this case, it is not necessary make the total length of the optical system remarkably long. Therefore, it is possible to secure the thickness of the positive lens L4.

In a case of exceeding an upper limit value of conditional expression (3), it is not possible to correct the curvature of field adequately, and a length of the first lens group G1 having a negative refractive power becomes long. Consequently, the total length of the optical system becomes excessively long. Therefore, it is not preferable to exceed the upper limit value of conditional expression (3).

In a case of falling below a lower limit value of conditional expression (3), a distance between lenses in the first lens group G1 having a negative refractive power becomes remarkably short. Therefore, it is necessary to make large the power of the first lens L1 having a negative refractive power particularly. As a result, various off-axis aberrations become susceptible to occur. Therefore, it is not preferable to fall below the lower limit value of conditional expression (3).

It is more preferable that the following conditional expression (3)′ be satisfied instead of conditional expression (3).

1.8≤D_1G/D_2T<3.0 (3)′

Furthermore, it is even more preferable that the following conditional expression (3)′ be satisfied instead of conditional expression (3).

2.0≤D_1G/D_2T<2.8 (3)′

Moreover, according to a preferable aspect of the present embodiment, it is preferable that the following conditional expression (4) be satisfied:

0.8<D_3G/D_2T<3.3 (4)

where,

D_3G denotes a thickness on the optical axis AX of the third lens group G3 having a positive refractive power, and

D_2T denotes the thickness on the optical axis AX of the positive lens L4 nearest to the image in the first lens group G1 having a negative refractive power.

Conditional expression (4) is related to an appropriate ratio of D_3G and D_2T. Moreover, conditional expression (4) is a conditional expression which regulates a ratio of the thickness on the optical axis AX of the third lens group G3 having a positive refractive power and the thickness of the positive lens L4 in the first lens group G1 having a negative refractive power.

Within a range satisfying conditional expression (4), correction of an axial aberration and a correction of an off-axis aberration become possible without the total length of the optical system becoming remarkably long.

In a case of exceeding an upper limit value of conditional expression (4), since the thickness of the positive lens L4 in the first lens group G1 having a negative refractive power becomes excessively thin, it is not possible to correct the off-axis aberration in particular. Therefore, it is preferable to exceed the upper limit value of conditional expression (4).

In a case of falling below a lower limit value of conditional expression (4), the thickness on the optical axis AX of the third lens group G3 having a positive refractive power becomes excessively thin, and it is not possible to correct the axial aberration in particular. Therefore, it is not preferable to fall below the lower limit value of conditional expression (4).

It is more preferable that the following conditional expression (4)′ be satisfied instead of conditional expression (4).

1.2<D_3G/D_2T<3 (4)′

Furthermore, it is even more preferable that the following conditional expression (4)′ be satisfied instead of conditional expression (4).

1.5<D_3G/D_2T<2 (4)′

Moreover, according to a preferable aspect of the present embodiment, it is preferable that the first lens group G1 having a negative refractive power include in order from the object side, a first lens L1 having a negative refractive power, and a cemented lens of a lens L3 having a negative refractive power and a lens L4 having a positive refractive power.

The first lens group G1 having a negative refractive power includes in order from the object side, the first lens L1 having a negative refractive power, and a cemented lens CL1 of the lens L3 having a negative refractive power and the lens L4 having a positive refractive power. Accordingly, it is possible to correct favorably a chromatic aberration even while securing a negative power which is necessary for achieving a long back focus. Therefore, it is preferable to make the abovementioned arrangement. A plane parallel plate L2 in FIG. 1 is a filter.

Examples of the present invention will be described below.

### EXAMPLE 1

Next, an objective optical system OBL which is included in an endoscope system 10 according to an example 1 will be described below.

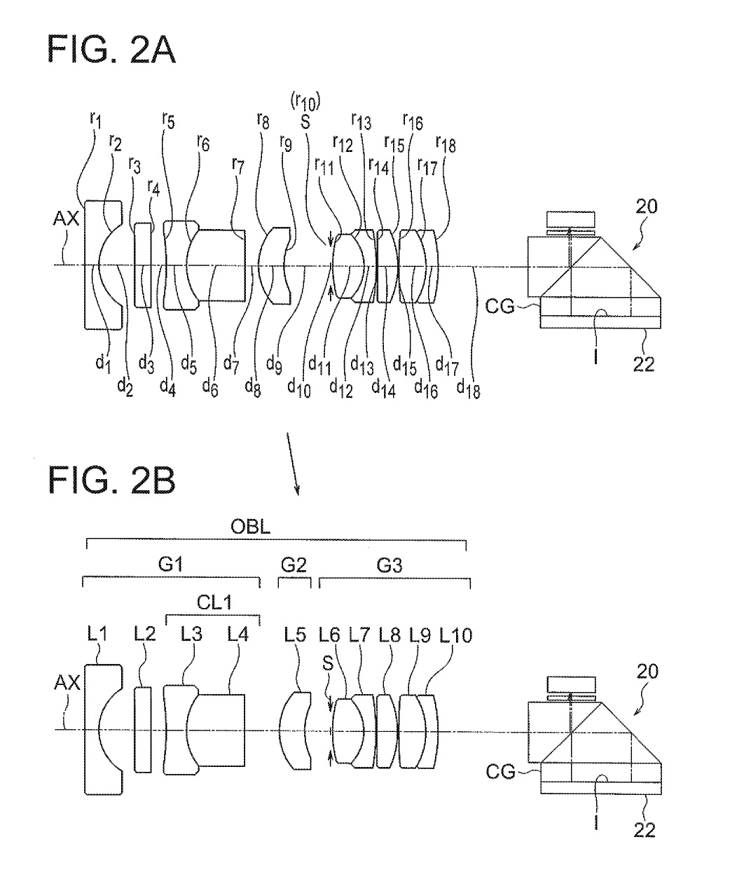

FIG. 2A and FIG. 2B are cross-sectional views of an arrangement of the objective optical system OBL. Here, FIG. 2A is a cross-sectional view of an arrangement of the objective optical system OBL in a normal observation state (an object point at a long distance), and FIG. 2B is a cross-sectional view of an arrangement of the objective optical system OBL in a close observation state (an object point at a close distance).

The objective optical system OBL according to the present example includes in order from an object side, a first lens group G1 having a negative refractive power, a second lens group G2 having a positive refractive power, and a third lens group G3 having a positive refractive power. Moreover, an aperture stop S is disposed in the third lens group G3. The second lens group G2 moves toward an image side on an optical axis AX, and corrects a change in a focal position due to a change from the normal observation state to the close observation state.

The first lens group G1 includes in order from the object side, a planoconcave negative lens L1 having a flat surface directed toward the object side, a plane parallel plate L2, a biconcave negative lens L3, and a positive meniscus lens L4 having a convex surface directed toward an image side. Here, the negative lens L3 and the positive meniscus lens L4 are cemented and form a cemented lens CL1.

The second lens group G2 includes a positive meniscus lens L5 having a convex surface directed toward the object side.

The third lens group G3 includes in order from the object side, the aperture stop S, a biconvex positive lens L6, a negative meniscus lens L7 having a convex surface directed toward the image side, a planoconvex positive lens L8 having a flat surface directed toward the object side, a biconvex positive lens L9, and a negative meniscus lens L10 having a convex surface directed toward the image side. Here, the positive lens L6 and the negative meniscus lens L7 are cemented. The positive lens L9 and the negative meniscus lens L10 are cemented.

An optical-path splitter 20, which will be described later, is disposed on the image side of the third lens group G3. In a prism in an optical system, an optical path is bent. The plane parallel plate L2 is a filter applied a coating for cutting specific wavelengths such as 1060 nm of YAG laser, 810 nm of semiconductor laser, or an infra-red region. Here, I is an image plane (image pickup surface).

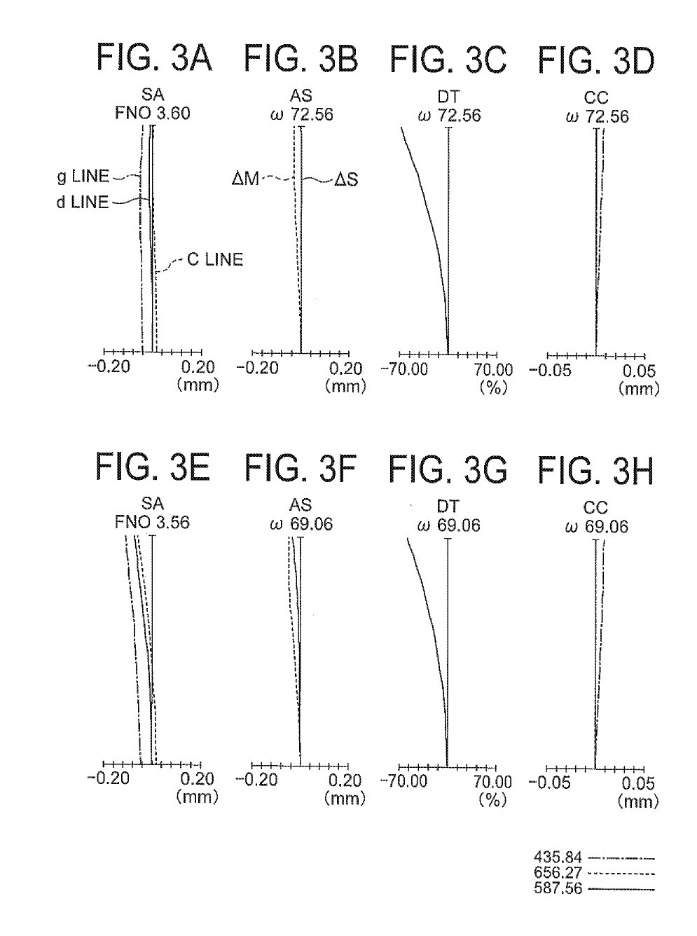

FIG. 3A, FIG. 3B, FIG. 3C, and FIG. 3D show a spherical aberration (SA), an astigmatism (AS), a distortion (DT), and a chromatic aberration of magnification (CC) respectively, in the normal observation state of the present example.

FIG. 3E, FIG. 3F, FIG. 3G, and FIG. 3H show a spherical aberration (SA), an astigmatism (AS), a distortion (DT), and a chromatic aberration of magnification (CC) respectively, in the close observation state of the present example.

In each of the aberration diagrams, a horizontal axis indicates an aberration amount. The unit of the aberration amount is mm for the spherical aberration, the astigmatism, and the chromatic aberration of magnification. Moreover, the unit of aberration amount is % for the distortion. Furthermore, FNO denotes an F-number. The unit of a wavelength of an aberration curve is nm. In various aberration diagrams, the aberration curves for the wavelengths of 656.27 nm (C-line), 587.56 nm (d-line), and 435.84 nm (g-line) are shown. Similar is a case in aberration diagrams of the following examples.

### EXAMPLE 2

Next, an objective optical system OBL which is included in an endoscope system 10 according to an example 2 will be described below.

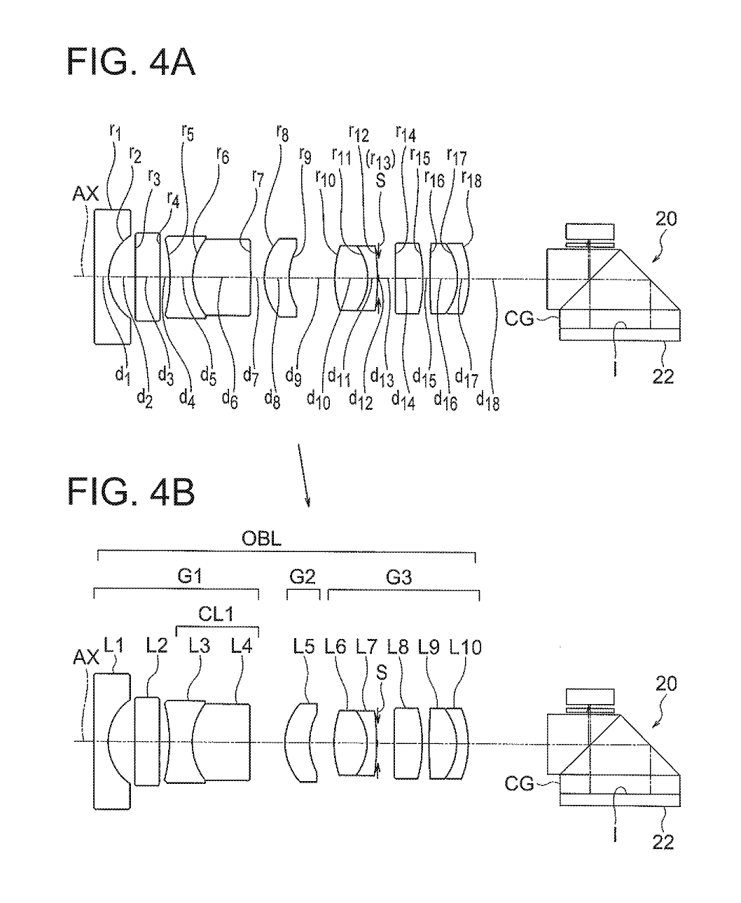

FIG. 4A and FIG. 4B are cross-sectional views of an arrangement of the objective optical system OBL. Here, FIG. 4A is a cross-sectional view of an arrangement of the objective optical system OBL in a normal observation state (an object point at a long distance), and FIG. 4B is a cross-sectional view of an arrangement of the objective optical system OBL in a close observation state (an object point at a close distance).

The objective optical system OBL according to the present example includes in order from an object side, a first lens group G1 having a negative refractive power, a second lens group G2 having a positive refractive power, and a third lens group G3 having a positive refractive power. Moreover, an aperture stop S is disposed in the third lens group G3. The second lens group G2 moves toward an image side on an optical axis AX, and corrects a change in a focal position due to a change from the normal observation state to the close observation state.

The first lens group G1 includes in order from the object side, a planoconcave negative lens L1 having a flat surface directed toward the object side, a plane parallel plate L2, a biconcave negative lens L3, and a biconvex positive lens L4. Here, the negative lens L3 and the positive lens L4 are cemented and form a cemented lens CL1.

The second lens group G2 includes a positive meniscus lens L5 having a convex surface directed toward the object side.

The third lens group G3 includes in order from the object side, a biconvex positive lens L6, a negative meniscus lens L7 having a convex surface directed toward an image side, the aperture stop S, a planoconvex positive lens L8 having a flat surface directed toward the object side, a biconvex positive lens L9, and a negative meniscus lens L10 having a convex surface directed toward the image side. Here, the positive lens L6 and the negative meniscus lens L7 are cemented. The positive lens L9 and the negative meniscus lens L10 are cemented.

An optical-path splitter 20, which will be described later, is disposed on the image side of the third lens group G3. In a prism in an optical system, an optical path is bent. The plane parallel plate L2 is a filter applied a coating for cutting specific wavelengths such as 1060 nm of YAG laser, 810 nm of semiconductor laser, or an infra-red region. Here, I is an image plane (image pickup surface).

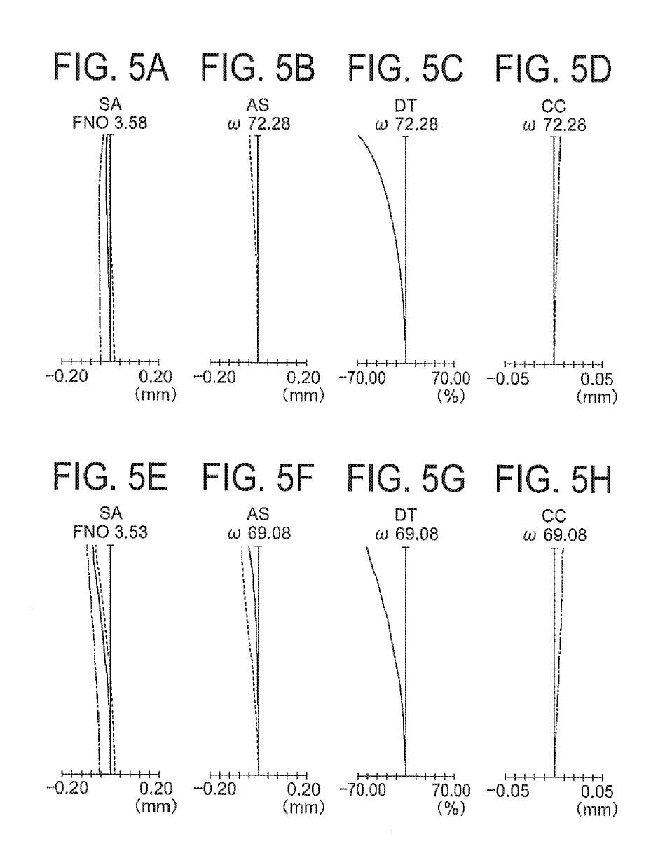

FIG. 5A, FIG. 5B, FIG. 5C, and FIG. 5D show a spherical aberration (SA), an astigmatism (AS), a distortion (DT), and a chromatic aberration of magnification (CC) respectively, in the normal observation state of the present example.

FIG. 5E, FIG. 5F, FIG. 5G, and FIG. 5H show a spherical aberration (SA), an astigmatism (AS), a distortion (DT), and a chromatic aberration of magnification (CC) respectively, in the close observation state of the present example.

### EXAMPLE 3

Next, an objective optical system OBL which is included in an endoscope system 10 according to an example 3 will be described below.

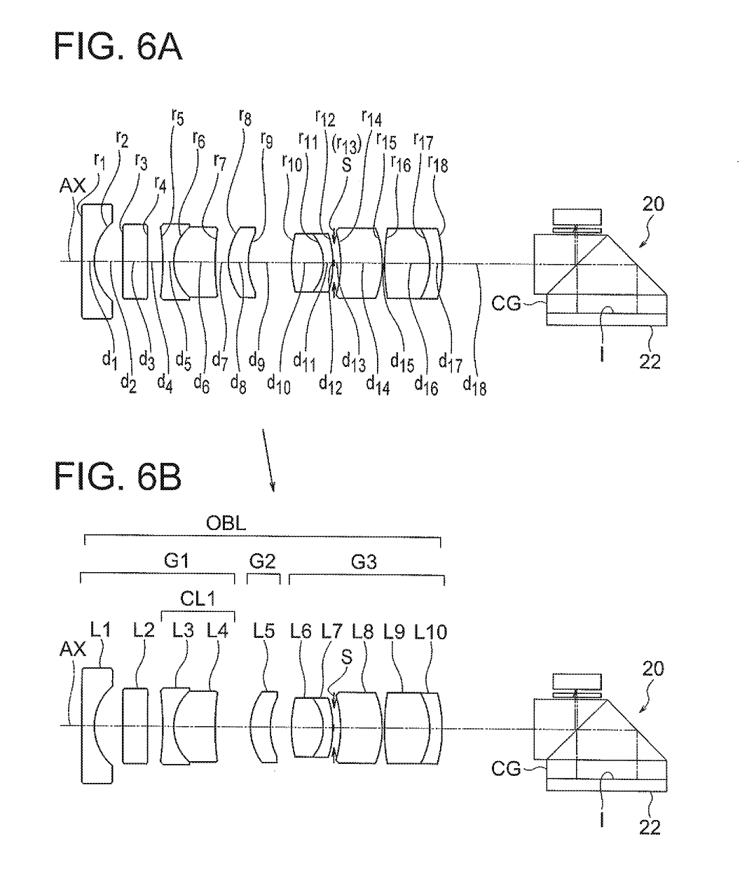

FIG. 6A and FIG. 6B are cross-sectional views of an arrangement of the objective optical system OBL. Here, FIG. 6A is a cross-sectional view of an arrangement of the objective optical system OBL in a normal observation state (an object point at a long distance), and FIG. 6B is a cross-sectional view of an arrangement of the objective optical system OBL in a close observation state (an object point at a close distance).

The objective optical system OBL according to the present example includes in order from an object side, a first lens group G1 having a negative refractive power, a second lens group G2 having a positive refractive power, and a third lens group G3 having a positive refractive power. Moreover, an aperture stop S is disposed in the third lens group G3. The second lens group G2 moves toward an image side on an optical axis AX, and corrects a change in a focal position due to a change from the normal observation state to the close observation state.

The first lens group G1 includes in order from an object side, a planoconcave negative lens L1 having a flat surface directed toward the object side, a plane parallel plate L2, a biconcave negative lens L3, and a positive meniscus lens L4 having a convex surface directed toward the object side. Here, the negative lens L3 and the positive meniscus lens L4 are cemented and form a cemented lens CL1.

The second lens group G2 includes a positive meniscus lens L5 having a convex surface directed toward the object side.

The third lens group G3 includes in order from the object side, a biconvex positive lens L6, a negative meniscus lens L7 having a convex surface directed toward an image side, the aperture stop S, a positive meniscus lens L8 having a convex surface directed toward the image side, a biconvex positive lens L9, and a negative meniscus lens L10 having a convex surface directed toward the image side. Here, the positive lens L6 and the negative meniscus lens L7 are cemented. The positive lens L9 and the negative meniscus lens L10 are cemented.

An optical path splitter 20, which will be described later, is disposed on the image side of the third lens group G3. In a prism in an optical system, an optical path is bent. The plane parallel plate L2 is a filter applied a coating for cutting specific wavelengths such as 1060 nm of YAG laser, 810 nm of semiconductor laser, or an infra-red region. Here, I is an image plane (image pickup surface).

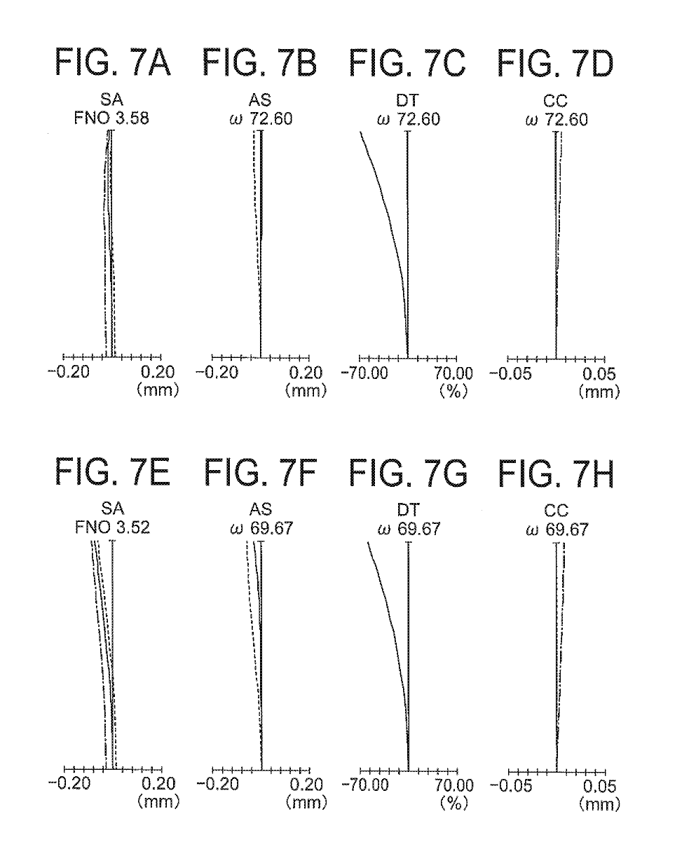

FIG. 7A, FIG. 7B, FIG. 7C, and FIG. 7D show a spherical aberration (SA), an astigmatism (AS), a distortion (DT), and a chromatic aberration of magnification (CC) respectively, in the normal observation state of the present example.

FIG. 7E, FIG. 7F, FIG. 7G, and FIG. 7H show a spherical aberration (SA), an astigmatism (AS), a distortion (DT), and a chromatic aberration of magnification (CC) respectively, in the close observation state of the present example.

Numerical data of each example described above is shown below. In symbols, r denotes radius of curvature of each lens surface, d denotes a distance between respective lens surfaces, nd denotes a refractive index of each lens for a d-line, νd denotes an Abbe number for each lens, f denotes a focal length, FNO denotes an F number, ω denotes a half angle of view, LTL denotes a lens total length. Moreover, the lens total length is a distance (not subjected to air conversion) from a frontmost lens surface to the rearmost lens surface plus back focus. The back focus is a unit which is expressed upon air conversion of a distance from a rearmost lens surface to a paraxial image surface.

### EXAMPLE 1

Unit mm
Surface data

| Surface no. | r | d | nd | νd |
|---|---|---|---|---|
| 1 | ∞ | 0.48 | 1.88300 | 40.76 |
| 2 | 1.654 | 1.20 | | |
| 3 | ∞ | 0.55 | 1.52100 | 65.12 |
| 4 | ∞ | 0.52 | | |
| 5 | −8.076 | 0.69 | 1.88300 | 40.76 |
| 6 | 1.959 | 1.98 | 1.84666 | 23.78 |
| 7 | ∞ | Variable | | |
| 8 | 1.959 | 0.87 | 1.48749 | 70.23 |
| 9 | 2.065 | Variable | | |
| 10 (Stop) | ∞ | 0.07 | | |
| 11 | 3.824 | 1.07 | 1.64769 | 33.79 |
| 12 | −1.556 | 0.41 | 2.00330 | 28.27 |
| 13 | −7.495 | 0.04 | | |
| 14 | ∞ | 0.69 | 1.69895 | 30.13 |
| 15 | −2.931 | 0.04 | | |
| 16 | 17.462 | 0.92 | 1.48749 | 70.23 |
| 17 | −2.333 | 0.41 | 1.92286 | 18.90 |
| 18 | −4.985 | 4.44 | | |
| 19 (Imaging surface) | ∞ | | | |

Zoom data

| | Normal observation | Close observation |
|---|---|---|
| | state | state |
| f | 1.00 | 1.00 |
| FNO. | 3.60 | 3.56 |
| 2ω | 145.13 | 138.11 |
| LTL (in air) | 16.43 | 16.43 |
| d7 | 0.45 | 1.17 |
| d9 | 1.59 | 0.88 |

Unit focal length

| | | |
|---|---|---|
| f1 = −1.23 | f2 = 21.18 | f3 = 3.27 |

### Example 2

Unit mm
Surface data

| Surface no. | r | d | nd | νd |
|---|---|---|---|---|
| 1 | ∞ | 0.49 | 1.88300 | 40.76 |
| 2 | 1.742 | 0.89 | | |
| 3 | ∞ | 0.84 | 1.52100 | 65.12 |
| 4 | ∞ | 0.34 | | |
| 5 | −4.963 | 0.77 | 1.88300 | 40.76 |
| 6 | 2.138 | 1.96 | 1.84666 | 23.78 |
| 7 | −59.859 | Variable | | |
| 8 | 1.999 | 0.84 | 1.48749 | 70.23 |
| 9 | 2.121 | Variable | | |
| 10 | 3.457 | 1.12 | 1.64769 | 33.79 |
| 11 | −1.805 | 0.32 | 2.00330 | 28.27 |
| 12 | −7.816 | 0.04 | | |
| 13 (Stop) | ∞ | 0.56 | | |
| 14 | 138.547 | 0.95 | 1.69895 | 30.13 |
| 15 | −4.718 | 0.24 | | |
| 16 | 19.967 | 0.94 | 1.48749 | 70.23 |
| 17 | −1.933 | 0.39 | 1.92286 | 18.90 |
| 18 | −3.202 | 4.53 | | |
| 19 (Imaging surface) | ∞ | | | |

Zoom data

| | Normal observation | Close observation |
|---|---|---|
| | state | state |
| f | 1.00 | 1.01 |
| FNO. | 3.58 | 3.53 |
| 2ω | 144.56 | 138.16 |
| LTL (in air) | 17.24 | 17.24 |
| d7 | 0.46 | 1.19 |
| d9 | 1.56 | 0.83 |

Unit focal length

| | | |
|---|---|---|
| f1 = −1.19 | f2 = 21.82 | f3 = 3.60 |

### Example 3

Unit mm
Surface data

| Surface no. | r | d | nd | νd |
|---|---|---|---|---|
| 1 | ∞ | 0.42 | 1.88300 | 40.76 |
| 2 | 1.805 | 0.96 | | |
| 3 | ∞ | 0.84 | 1.52100 | 65.12 |
| 4 | ∞ | 0.51 | | |
| 5 | −8.465 | 0.37 | 1.88300 | 40.76 |
| 6 | 1.614 | 1.40 | 1.84666 | 23.78 |
| 7 | 9.296 | Variable | | |
| 8 | 2.036 | 0.67 | 1.48749 | 70.23 |
| 9 | 2.255 | Variable | | |
| 10 | 3.940 | 1.09 | 1.64769 | 33.79 |
| 11 | −1.532 | 0.32 | 2.00330 | 28.27 |
| 12 | −3.309 | 0.04 | | |
| 13 (Stop) | ∞ | 0.23 | | |
| 14 | −3.767 | 1.41 | 1.77250 | 49.60 |
| 15 | −3.239 | 0.07 | | |
| 16 | 7.254 | 1.55 | 1.48749 | 70.23 |
| 17 | −2.390 | 0.42 | 1.92286 | 18.90 |
| 18 | −4.506 | 4.52 | | |
| 19 (Imaging surface) | ∞ | | | |

Zoom data

| | Normal observation | Close observation |
|---|---|---|
| | state | state |
| f | 1.00 | 1.01 |
| FNO. | 3.58 | 3.52 |
| 2ω | 145.20 | 139.33 |
| LTL (in air) | 16.74 | 16.74 |
| d7 | 0.45 | 1.20 |
| d9 | 1.47 | 0.72 |

Unit focal length

| | | |
|---|---|---|
| f1 = −1.01 | f2 = 21.36 | f3 = 3.33 |

Values of conditional expressions in each example are shown below.

| | Example 1 | Example 2 | Example 3 |
|---|---|---|---|
| (1) D_2T/fw | 1.98 | 1.96 | 1.40 |
| (2) ω (wide)/ω (tele) | 1.05 | 1.05 | 1.04 |
| (3) D_1G/D_2T | 2.74 | 2.70 | 3.21 |
| (4) D_3G/D_2T | 1.85 | 2.33 | 3.66 |

Values of parameters are given below.

| | Example 1 | Example 2 | Example 3 |
|---|---|---|---|
| D_2T | 1.98 | 1.96 | 1.40 |
| D_1G | 5.42 | 5.29 | 4.51 |
| D_3G | 3.66 | 4.56 | 5.13 |
| fw | 1.00 | 1.00 | 1.00 |
| ω (wide) | 72.57 | 72.28 | 72.60 |
| ω (tele) | 69.06 | 69.08 | 69.67 |

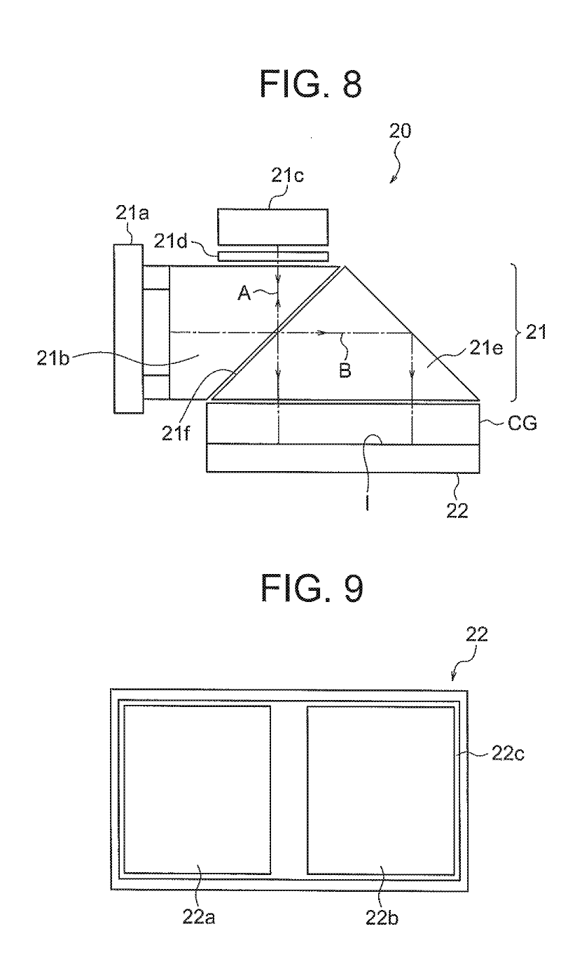

An arrangement of the optical-path splitter 20 will be described below. FIG. 8 is a diagram showing a schematic arrangement of the optical-path splitter 20 and an image pickup element 22.

Light emerged from the objective optical system OBL is incident on the optical-path splitter 20.

The optical-path splitter 20 includes a polarization beam splitter 21 which splits an object image into two optical images with different focus, and the image pickup element 22 which acquires two images by capturing the two optical images.

The polarization beam splitter 21, as shown in FIG. 8, includes a prism 21b on the object side, a prism 21e on the image side, a mirror 21c, and a λ/4 plate 21d. Both the prism 21b on the object side (the object-side prism) and the prism 21e on the image side (the image-side prism) have a beam splitting surface which is inclined at 45 degrees with respect to the optical axis AX.

A polarization splitting film 21f is formed on the beam splitting surface of the prism 21b on the object side. Moreover, in the prism 21b on the object side and the prism 21e on the image side, the beam splitting surfaces are in mutual contact via the polarization splitting film 21f. The polarization beam splitter 21 is formed in such manner.

Moreover, the mirror 21c is provided near an end surface of the prism 21b on the object side via the λ/4 plate 21d. The image pickup element 22 is attached to an end surface of the prism 21e on the image side via a cover glass CG. Here, I is an image plane (image pickup surface).

Light of an object image emerges from the objective optical system OBL. The light emerged is split into a P-polarized component (light transmitted) and an S-polarized component (light reflected) in the prism 21b on the object side. The splitting occurs due to the polarization splitting film 21f provided to a beam-splitting surface. As a result, the object image is divided into two optical images which are an optical image on a side of light reflected and an optical image on a side of light transmitted.

An optical image of the S-polarized component is reflected at the polarization splitting film 21f toward a side facing the image pickup element. The optical image reflected travels an optical path A, and is transmitted through the λ/4 plate 21d. At this time, in the optical image, a direction of polarization rotates through 90°. The optical image is reflected at the mirror 21c, and is returned toward a side of the image pickup element 22. By the optical image returned being transmitted once again through the λ/4 plate 21d, in the the optical image, the direction of polarization rotates through 90°. The optical image is transmitted through the polarization splitting film 21f, and is formed on the image pickup element.

An optical image of the P-polarized component is transmitted through the polarization splitting film 21f, and travels an optical path B. The optical path B is formed by the prism 21e on the image side which returns perpendicularly toward the image pickup element 22. The prism 21e has a mirror surface which is provided on an opposite side of the beam-splitting surface. The optical image of the P-polarized component is reflected by the mirror surface, and is formed as an image on the image pickup element 22. At this time, a path in a prism glass is to be set such that a predetermined optical path difference of about several tens of μm occurs between the optical path A and the optical path B, and two optical images with different focus are formed on a light receiving surface of the image pickup element 22.

In other words, an arrangement is to be made such that it is possible to split the object image into two optical images with different focus position in the prism 21b on the object side and the prism 21e on the image side. For instance, an arrangement is to be made such that an optical path length on a side of the light reflected becomes short (small) with respect to an optical path length (path length in glass) on a side of the light transmitted which reaches the image pickup element 22 in the prism 21b on the object side.

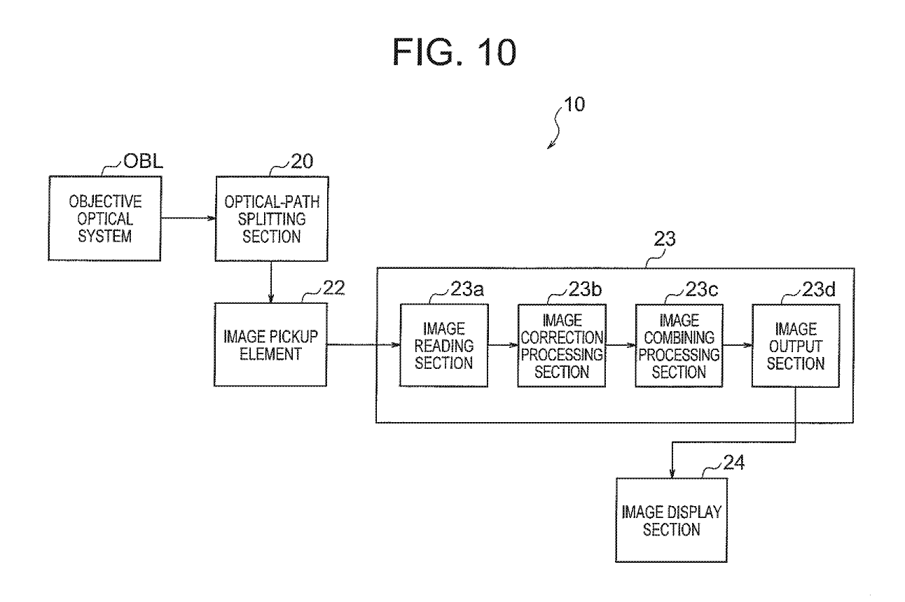

FIG. 9 is a schematic block diagram of the image pickup element 22. The image pickup element 22, as shown in FIG. 9, is provided with two light receiving areas (effective pixel areas) 22a and 22b in an overall pixel area of the image pickup element 22, for receiving separately and capturing the two optical images with different focusing position.

The light receiving areas 22a and 22b are disposed to coincide with an image forming surfaces of the optical images respectively for capturing two optical images. Moreover, in the image pickup element 22, the light receiving area 22a has a focusing position thereof with respect to the light receiving area 22b, shifted relatively toward a near-point side, and the light receiving area 22b has a focusing position thereof with respect to the light receiving area 22a, shifted relatively toward a far-point side. Accordingly, an arrangement is made to form two optical images with different focus on the light receiving surface of the image pickup element 22.

An arrangement may be made such that an optical path length up to the image pickup element 22 is changed by using a glass material for the prism 21b on the object side and a glass material for the prism 21e on the image side such that, a refractive index of the glass material of the prism 21b on the object side differs from a refractive index of the glass material of the prism 21e on the image side, and the focusing position with respect to the light receiving areas 22a and 22b is shifted relatively.

Moreover, a correction pixel area 22 for correcting a geometrical shift of the two split optical images is provided around the light receiving areas 22a and 22b. The abovementioned geometrical shift in the optical images is to be eliminated by suppressing a manufacturing error within the correction pixel area 22c and carrying out a correction by image processing in an image correction processing section 23b (FIG. 10) which will be described later.

Moreover, as shown in FIG. 1, the abovementioned second lens group G2 in the present embodiment is a focusing lens, and can be moved selectively to two positions in a direction of the optical axis. The second lens group G2 is moved from one of the two positions to the other position and vice versa by an actuator which is not shown in the diagram.

In a state where the second lens group G2 is set to a position on a frontward side (object side), the second lens group G2 is set such that an object in an observation area in a case of a distant observation (normal observation) is focused. Moreover, in a state where the second lens group G2 is set to a position on a rearward side, the second lens group G2 is set such that an object in an observation area in a case of a close observation (magnified observation) is focused.

As in the present embodiment, in a case where polarization splitting is achieved by using the polarization beam splitter 21, when a state of polarization of light to be split is not a circularly-polarized state, a difference occurs in brightness of images that are split. Although correction of a difference in brightness occurring regularly is comparatively easier, when the difference in brightness occurs locally and due to observation conditions, it is not possible to correct the difference in brightness, and an uneven brightness may occur in a combined image.

In an image such as a combined image of an object observed by an endoscope, there is a possibility that the unevenness in brightness relatively occurs in a periphery portion of a field of view. The unevenness in brightness in which the polarization state is disrupted occurs remarkably when the object has a brightness distribution with a relatively saturated tendency.

In an endoscope, the periphery portion of the field of view is relatively closer to an object. Therefore, in the periphery portion of the field of view, a blood stream and a mucosal integrity is seen often. There is a high possibility that an image in which the unevenness in brightness has occurred in the periphery portion of the field of view becomes an image which is extremely complicated for a user.

Therefore, for example, it is preferable to dispose the λ/4 plate 21a on the object side of the polarization splitting film 21f of the optical-path splitter 20 as shown in FIG. 8, in order to return the disrupted polarization state to a circularly-polarized state.

It is also possible to use a half mirror which splits an intensity of incident light, instead of the abovementioned polarization beam splitter 21.

In the present embodiment, by generating a combined image in which the two optical images with different focus are acquired, it becomes possible to achieve an image with a wide depth of field. Moreover, the optical-path splitter 20 includes the two prisms 21b and 21e. An image is split into two by using the prisms 21b and 21e, and the two images are taken in by one image pickup element 22. By doing this, since only one image pickup element 22 is used, the cost becomes cheaper, and it is preferable.

Next, the combining of two images that are taken in will be described below by referring to FIG. 10. FIG. 10 is a functional block diagram of the endoscope system 10.

An image processor 23 has an image reading section 23a, the image correction processing section 23b, the image combining processing section 23c, and an image output section 23d. The image reading section 23a reads each of images related to the two optical images with different focusing position captured by the image pickup element 22. The image correction processing section 23b carries out image correction of two images read by the image reading section 23a. The image combining processing section 23c carries out image combining processing of combining the two images that have been corrected. The image output section 23d outputs the image combined by the image combining processing section 23c.

An optical image is formed on each of the light receiving areas 22a and 22b of the image pickup element 22. The image correction processing section 23b corrects the images related to two optical images formed such that a mutual difference, except for the focusing, becomes substantially same. In other words, the correction of two images is carried out such that a relative position, a relative angle, and a relative magnification for each optical image of the two images become substantially same.

In a case of splitting an object image into two, and forming each image on the image pickup element 22, a geometrical difference may occur. In other words, in the optical image formed on each of the light receiving areas 22a and 22b (FIG. 9) of the image pickup element 22, shift in magnification, shift in position, and shift in angle, or in other words, shift in a direction of rotation may relatively occur for the two optical images.

It is difficult to eliminate entirely these shifts at a time of manufacturing. However, these shifts remained, when particularly the amount of shift becomes large, the combined image becomes a double image and an unnatural unevenness in brightness occurs. Therefore, the abovementioned geometrical difference and the difference in brightness are to be corrected in the image correction processing section 23b.

In a case of correcting the difference in brightness of the two images, it is desirable to carry out correction with reference to an image with a low brightness out of the two images, or to an image for which the brightness at the same position is relatively low out of the two images. Two optical images may be used instead of two imaged.

The image combining processing section 23c generates a combined image by selecting an image with a comparatively high contrast, in a corresponding predetermined area between the two images corrected by the image correction processing section 23b. In other words, the image correction processing section 23c compares the contrast in each of the spatially same pixel areas in the two images, and by selecting the pixel area in which the contrast is comparatively higher, and generates a combined image as an image in which the two images are combined.

In a case in which a difference in contrast in the same pixel area of two images is either small or substantially same, a combined image is generated by image combining processing in which a predetermined weighting of that pixel area is carried out and then added.

Moreover, the image processor 23 carries out post image processing such as color matrix processing, outline enhancement, and gamma correction on one image combined by the image combining processing section 23c. The image output section 23d outputs an image which is subjected to the post image processing. The image output from the image output section 23d is output to an image display section 24.

Moreover, the relative focusing position may be shifted by making the prism 21b on the object side and the prism 21e on the image side of different glass materials, and making a refractive index to differ, in accordance with a near-point optical path and a far-point optical path reaching the image pickup element 22.

Accordingly, it is possible to achieve a combined depth of field by acquiring images related to two optical images with different focus and combining the acquired images by the image combining processing section 23c. In an endoscopic inspection, the distant observation (the normal observation) is appropriate at a time of screening by taking a long shot of a wide range, and the close observation (the magnified observation) is appropriate at a time of observing details of a pathologic lesion or making a diagnosis.

By making such arrangement, it becomes possible to widen the depth of field without degradation of resolving power even when an image pickup element in which the number of pixels is made further larger is used. Furthermore, since there is a focusing mechanism, by switching the observation range freely, it is possible to carry out endoscope observation with a high image quality, and diagnosis.

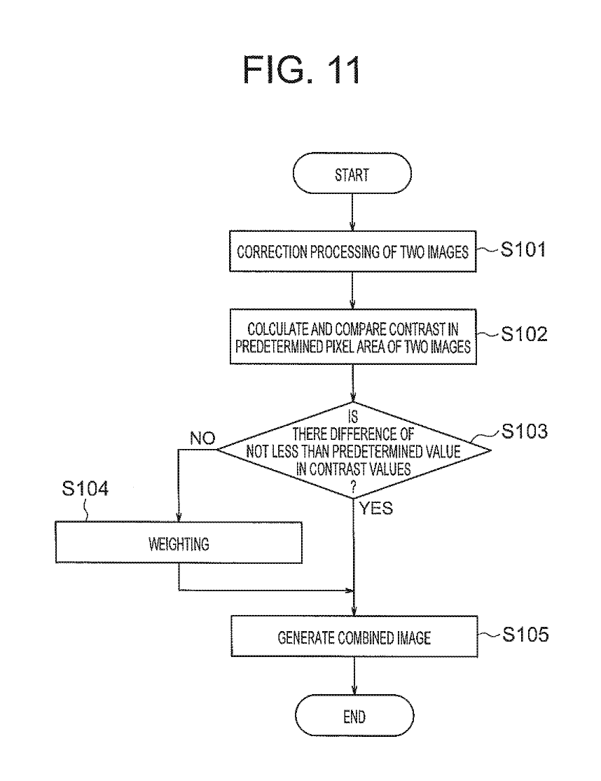

Next, in the present embodiment, a flow in a case of combining two optical images will be described below according to a flowchart in FIG. 11.

An image related to the far-point image and an image related to the near-point image with a different focus are acquired in the image pickup element 22. At step S101, the two images which are the near-point image and the far-point image, are subjected to correction processing. In other words, according to correction parameters that have been set in advance, correction of two images is carried out such that the relative position, the relative angle, and the relative magnification of each optical image of the two images becomes substantially same. This correction processing is carried out in the image correction processing section 23b. Images after correction are output to the image combining processing section 23c. The brightness and color of the two images may be corrected according to the requirement.

At step S102, for the two images subjected to the correction processing, or in other words, for the pixel area corresponding to each of the far-point image and the near-point image, a contrast value is calculated, and the contrast values are compared.

At step S103, a judgment of whether or not there is a difference in the contrast values that have been compared is made.

In a case in which there is a difference in the contrast, the process advances to step S105. In a case in which there is no difference in the contrast or in a case in which the difference in the contrast is small, the process advances to step S104.

At step S105, the image combining is carried out. In a case in which there is a difference in the contrast, an area with a high contrast value is selected, and the images are combined.

In a case in which the difference in the contrast values is small or in a case in which the contrast values are almost same, it is necessary to make a judgment which to select between the two images which are the far-point image and the near point image. Wrong choice of the selection becomes a cause of unstable processing. For instance, in a case in which a selected image includes a fluctuation in a signal such as noise, a discontinuous area occurs in the combined image or a problem such that an object image which is resolved originally becomes blurred occurs.

Therefore, the process advances to step S104 and the weighting is carried out. At step S104, in the pixel area in which the contrast is compared, in a case in which the contrast values for the two images which are the far-point image and the near-point image are almost same, the weighting is carried out. Moreover, the instability of the image selection is eliminated by carrying out an addition processing of images subjected to weighting at the subsequent step S105.

In such manner, according to the present embodiment, in both the close observation and the distant observation, it is possible to acquire an image in which the depth of field has been widened, while preventing the blurring of the optical image and the occurrence of the discontinuous area in the combined image due to noise.

Moreover, since both the images are captured by the same image pickup element, it is possible to reduce a manufacturing cost and to acquire an image in which the depth of field is widened without making an apparatus large in size, as compared to an objective optical system which includes a plurality of image pickup elements.

Moreover, it is possible to achieve the depth of field which is desired, and to prevent degradation of resolving power.

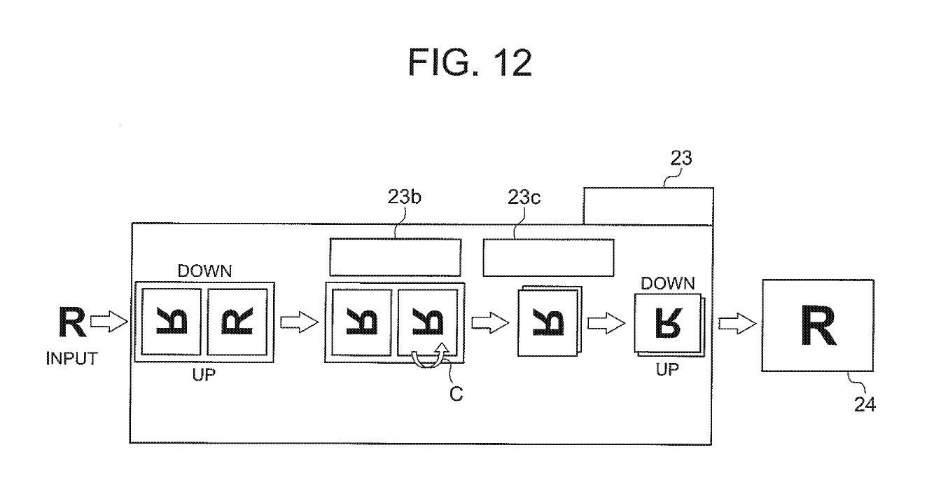

FIG. 12 is a diagram showing an image-formation state in a case in which an image is formed on an image pickup element after reflection for odd number of times by the polarization beam splitter 21. In a case of the abovementioned polarization beam splitter 21 in FIG. 8, an optical image is formed on the image pickup element 22 after one reflection or in other words after reflection for the odd number of times. Consequently, one of the two images assumes an image-formation state (mirror image) as shown in FIG. 12, and an image processing in which an image direction is made to coincide by inverting the mirror image in the image processor 23, is carried out.

Since correction of the mirror image by an optical reflection for the even number of times may lead to making the objective optical system large-size and the cost of the prism high, it is preferable to carryout the correction of the mirror image by reflection for the odd number of times by inverting the mirror image in the image correction processing section 23b.

In a case in which the image pickup element 22 has a shape which is long in a longitudinal direction of the endoscope, it is preferable to rotate the combined image appropriately up on taking into consideration an aspect ratio of the image display section 24.

The abovementioned objective optical system may satisfy the plurality of arrangements simultaneously. Making such arrangement is preferable for achieving a favorable objective optical system and an endoscope system. Moreover, a combination of the preferable arrangements is arbitrary. Furthermore, regarding conditional expressions, only an upper limit value or a lower limit value of a further restricted numerical range of the conditional expression may be restricted.

Various embodiments of the present invention have been described heretofore. However, the present invention is not restricted to the embodiments described heretofore, and embodiments in which arrangements of the abovementioned embodiments are combined appropriately without departing from the scope of the present invention also fall under the category of the present invention.

The present embodiment shows an effect that it is possible to provide an endoscope system having an objective optical system which enables to correct favorably the chromatic aberration of magnification by using a cemented lens in the first lens group having a negative refractive power, and to correct favorably an off-axis aberration, and particularly the curvature of field, by making thick the thickness of the lens having a positive refractive power.

As described heretofore, the present invention is useful for an endoscope system having an objective optical system which enables to correct favorably the chromatic aberration of magnification by using a cemented lens in the first lens group having a negative refractive power, and to correct favorably an off-axis aberration, and particularly the curvature of field, by making thick the thickness of the lens having a positive refractive power.

What is claimed is:

1. An endoscope system, comprising:
an objective optical system;
an optical-path splitter which splits an object image acquired by the objective optical system into two optical images with different focus by using two prisms; and
an image sensor which acquires the optical images;
wherein
the objective optical system includes in order from the object side to an image side,
a first lens group having a negative refractive power, which is fixed,
a second lens group having a positive refractive power, which is movable,
a third lens group having a positive refractive power, which is fixed, and
switching to a normal observation and a close observation is possible by moving the second lens group toward an image side,
the first lens group includes in order from the object side to the image side, a first lens having a negative refractive power, and at least one positive lens, and
the objective optical system satisfies the following conditional expressions (1)′″, (2), and (3)′″:
1.40≤D_2T/fw<5 (1)′″
1.01<ω(wide)/ω(tele)<3.0 (2)
2.70≤D_1G/D_2T<3.21 (3)′″
where,
D_2T denotes a thickness on an optical axis of the positive lens nearest to an image in the first lens group having a negative refractive power,
fw denotes a focal length of the objective optical system in a normal observation state,
ω(wide) denotes an angle of view of the objective optical system in the normal observation state,
ω(tele) denotes an angle of view of the objective optical system in a close observation state,
D_1G denotes a thickness on the optical axis of the first lens group having a negative refractive power, and
D_2T denotes a thickness on the optical axis of the positive lens nearest to the image in the first lens group having a negative refractive power.

2. The endoscope system according to claim 1, wherein the following conditional expression (4) is satisfied:
0.8<D_3G/D_2T<3.3 (4)
where,
D_3G denotes a thickness on the optical axis of the third lens group having a positive refractive power, and
D_2T denotes the thickness on the optical axis of the positive lens nearest to the image in the first lens group having a negative refractive power.

3. The endoscope system according to claim 1, wherein the first lens group having a negative refractive power includes in order from the object side to the image side, a first lens having a negative refractive power, and a cemented lens of a lens having a negative refractive power and a lens having a positive refractive power.
